(19) United States
(12) Patent Application Publication
(10) Pub. No.: US 2009/0292021 A1
(43) Pub. Date: Nov. 26, 2009

(54) ANTICONVULSIVE PHARMACEUTICAL COMPOSITIONS

(75) Inventors: Serge Picaud, Avon (FR); Agnes Duboc, Strasbourg (FR); Michel Paques, Paris (FR); Jose Sahel, Paris (FR); Manuel Simonutti, Paris (FR)

Correspondence Address: YOUNG & THOMPSON, 209 Madison Street, Suite 500, ALEXANDRIA, VA, 22314, US

(73) Assignee: INSERM (INSTITUT NATIONAL DE LA SANTE ET DE LA REC, PARIS (FR)

(21) Appl. No.: 12/375,970

(22) PCT Filed: Jul. 31, 2007
(86) PCT No.: PCT/EP07/57860

§ 371 Date: May 1, 2009

Related U.S. Application Data
(60) Provisional application No. 60/834,792, filed on Aug. 2, 2006.

(51) Int. Cl. A61K 31/197; A61P 25/08

(52) U.S. Cl. 514/561

(57) ABSTRACT

Pharmaceutical compositions including, as the active ingredients, a combination of vigabatrin and of at least one substance having anti-ischemic effect, are disclosed.

### FIELD OF THE INVENTION

The present invention relates to anticonvulsive pharmaceutical compositions comprising the anticonvulsive agent vigabatrin having reduced undesirable effects.

### BACKGROUND OF THE INVENTION

Epilepsy is a general term describing a group of central nervous system disorders that are characterized by recurrent seizures that are the outward manifestation of excessive and/or hyper-synchronous abnormal electrical activity of neurons of the cerebral cortex and other regions of the brain. This abnormal electrical activity can be manifested as motor, convulsion, sensory, autonomic, or psychic symptoms.

Epilepsy is one of the more common neurological disorders and affects millions of people worldwide, and over 2.5 million individuals in the United States.

It is believed that the characteristic seizures of epilepsy are caused by the disordered, synchronous, and rhythmic firing of brain neurons. The neurons can fire at up to four times their normal rate. As a result, epileptic seizures are an overstimulation of the normal neuronal processes that control brain function.

Even though existing antiepileptic drugs can render 80% of newly diagnosed patients seizure free, a significant number of patients have chronic intractable epilepsy causing disability with considerable socioeconomic implications.

Anti-epileptic drugs are available for treating epilepsies, but these agents have a number of shortcomings. For instance, the agents are often poorly soluble in aqueous and biological fluids or are extremely hygroscopic. Of even greater importance is that patients often become refractory to a drug over time. In addition, many anti-epileptic agents cause unwanted side effects, neurotoxicities, and drug interactions. Even while being treated with one or a combination of the anti-epileptic drugs currently in clinical use, 30% of epileptic patients still experience seizures. As more anti-epileptic drugs are developed, the clinician will have expanded pharmaceutical options when designing an effective treatment protocol for each patient.

Vigabatrin, which was developed as an inhibitor of gamma-aminobutyric acid transaminase, was one of the most promising novel anticonvulsant active ingredients. However, vigabatrin was shown to induce highly severe undesirable effects, such as an irreversible restriction of the visual field. The restriction of the visual field induced by vigabatrin is asymptomatic when it is restricted to the nasal quadrant, until it extends to more central areas. Furthermore, visual defects induced by vigabatrin are not limited to the constriction of the visual field but also includes dysfunction of central vision with a reduction of visual acuity, a loss of color discrimination and of contrast sensitivity. An arrest of a therapeutical treatment with vigabatrin allows a stabilization of the visual loss but very rarely induces any recovery.

However, because epileptic seizures are always very handicapping and may be lethal, vigabatrin is still prescribed.

There is thus a need in the art for improved anticonvulsive, including anti-epileptic, pharmaceutical compositions comprising vigabatrin, which would be endowed with reduced or no undesirable effects.

### SUMMARY OF THE INVENTION

It is provided according to the present invention novel anticonvulsive pharmaceutical compositions comprising vigabatrin and which possess reduced undesirable effects, as compared with the vigabatrin-based pharmaceutical compositions known in the art.

One object of the present invention consists of a pharmaceutical composition comprising, as the active ingredients, a combination of vigabatrin and of at least one substance having anti-ischemic effect.

This invention also pertains to a method for treating convulsive disorders, including epilepsy, comprising a step of administering, to a patient in need thereof, a combination of vigabatrin and of at least one substance having an anti-ischemic effect.

According to the present invention, substances having an anti-ischemic effect encompass antioxidant substances, free radical scavenger substances, statins, ACE inhibitors, AT-1 antagonists, calcium channel inhibitors, sodium channel blocker agents, potassium channel activator agents, beta-adrenergic blocking agents, inhibitors of the synaptic release of glutamate, antagonists of a glutamate receptor, anesthetics substances, anticonvulsive agents, NMDA-receptor antagonists, hormones, vasodilatator agents, α-receptor antagonists, xanthine oxidase inhibitors, cyclooxygenase inhibitors, protease inhibitors, immunosuppressant agents and mitochondrial ATP sensitive potassium channel opener agents.

### BRIEF DESCRIPTION OF THE FIGURES

FIG. 1: Dorso-ventral distribution of the vigabatrin-elicited damage. The graph provides the cumulative length of disorganized outer nuclear layer (ONL; Abscissa) with photoreceptor nuclei abutting the retinal pigment epithelium in the ventral (open square) and dorsal (dark square) areas for individual animals. Animals (Ordinate) are ordered in increasing lengths for the ventral area showing thereby high correlation (r²=0.885) between the extents of retinal damage in these ventral and dorsal areas. The dorsal retinal damage was always greater as indicated by individual animals and by the mean damaged areas (dorsal area: dotted line, ventral area: dashed line)

FIG. 2: Quantification of cone photoreceptor loss in vigabatrin-treated rats. Cones were counted on retinal sections labelled with the cone arrestin antibody in control animals and vigabatrin-treated mice.

Abscissa: (i) left bar: Control animals, (ii) right bar: Vigabatrin (VGB)-treated mice;
Ordinate: cone density per 300 μm;
Vigabatrin-treated animals exhibited a significant decrease (18.7%) in the number of labelled cone photoreceptors (P<0.01).

### DETAILED DESCRIPTION OF THE INVENTION

Surprisingly, it has been found according to the present invention that the progressive and irreversible loss of visual acuity of patients treated with vigabatrin is caused by the induction of an ischemia of the retinal tissue, which ischemia of the retina tissue leads to an irreversible loss of the cone function, associated with a major disorganisation of the outer nuclear layer, mainly localized in the upper part of the retina.

The occurrence of a retinal ischemia caused by a treatment with vigabatrin is herein demonstrated by the high similarities between the kind of cellular modifications which is seen in vigabatrin-treated animals, as compared to the cellular disorganization which was observed in animals subject to retinal detachment.

The occurrence of a retinal ischemia that is induced by an anticonvulsive treatment with vigabatrin has been further demonstrated by the showing of an increased expression of VEGF in glial cells throughout retina, as compared with non treated control animals. Notably, the increase in VEGF expression that is shown herein clearly illustrates the vigabatrin-induced defect in the blood feeding to photoreceptors and the concomitant VEGF-induced tentative of neovascularisation of the retina.

These surprising findings have allowed the inventors to design improved vigabatrin-based anticonvulsive pharmaceutical compositions having reduced side effects as compared to the pharmaceutical compositions comprising vigabatrin that are already known in the art, the said improved pharmaceutical compositions comprising, additionally to vigabatrin, also at least one substance that is effective against ischemia.

Vigabatrin consists of the International Common Name of 4-amino-5-hexenoic acid, which may also be termed 4-aminohex-5-enoic acid (IUPAC designation), which chemical formula is C₆H₁₁NO₂, and which has the CAS Registry accession number 60643-86-9.

Thus, one object of the present invention consists of an anticonvulsive pharmaceutical composition comprising, as the active ingredients, a combination of (i) 4-amino-5-hexenoic acid and (ii) at least one substance having an anti-ischemic effect.

By the expression “at least one substance”, it is intended herein “one or more substance(s)”.

As used herein, an anticonvulsive pharmaceutical composition consists of a pharmaceutical composition that is active for preventing and treating convulsive disorders. Convulsive disorders encompass epilepsy, tuberous sclerosis, as well as the convulsive disorders affecting patients undergoing a drug addiction, including a drug addiction to heroin or cocaine.

As used herein, a substance having an effect against ischemia, which may also be termed an anti-ischemic substance or a substance having an anti-ischemic effect, encompasses notably those substances that are active against retinal ischemia.

As used herein, “retinal ischemia” encompasses generally any disorder involving an hypoxic condition of the retinal tissue, especially any disorder that reduces availability of blood, oxygen or other nutrients to the retinal tissue and which can result in retinal tissue disorganization and retinal cell death. Thus, retinal ischemia encompasses hypoxic conditions of the retinal tissue, notably those that are caused by a reduction of the arterial blood flow or the venous blood flow to, or in, the retina, as well as those resulting from a dysfunction of the retinal pigment epithelium in transferring glucose and oxygen to photoreceptors.

Thus, in an anticonvulsive pharmaceutical composition according to the invention, vigabatrin may be combined to any substance that possesses an activity against ischemia. Notably, in an anticonvulsive pharmaceutical composition according to the invention, vigabatrin may be combined with any one of those substances that are already known to exert an anti-ischemic effect.

In an anticonvulsive pharmaceutical composition according to the invention, the active ingredients consist of the combination of vigabatrin with at least one substance having an anti-ischemic effect.

In certain embodiments of an anticonvulsive pharmaceutical composition according to the invention, the said substance having an anti-ischemic effect is selected from the group consisting of an antioxidant substance, a free radical scavenger substance, a statin, an ACE inhibitor, an AT-1 antagonist, a calcium channel inhibitor, a sodium channel blocker agent, a potassium channel activator agent, a beta-adrenergic blocking agent, an inhibitor of the synaptic release of glutamate, an antagonist of a glutamate receptor, an anesthetics substance, an anticonvulsive agent, an NMDA-receptor antagonist, a hormone, a vasodilatator agent, an α-receptor antagonist, a xanthine oxidase inhibitor, a cyclooxygenase inhibitor, a protease inhibitor, an immunosuppressant agent and a mitochondrial ATP sensitive potassium opener agent.

An anticonvulsive pharmaceutical composition according to the invention may comprise more than one anti-ischemic substance, so as to further reduce the deleterious effect of vigabatrin on the retinal tissue.

Thus, in certain embodiments of an anticonvulsive pharmaceutical composition as described herein, the said composition may comprise, additionally to vigabatrin, 2, 3, 4, 5, 6, 7, 8, 9 or 10 distinct anti-ischemic substances, especially among those belonging to the various classes of anti-ischemic substances that are listed above.

In certain embodiments, each of the more than one anti-ischemic substance that is comprised in an anticonvulsive pharmaceutical composition according to the invention belongs to a specific class of anti-ischemic substances, which class is distinct from the classes to which belong the at least one other anti-ischemic substance combined therewith.

However, in most embodiments, an anticonvulsive pharmaceutical composition according to the present invention comprises, in combination to vigabatrin, 1, 2 or 3 distinct anti-ischemic substances, wherein, optionally, each of the anti-ischemic substance belongs to a specific class of anti-ischemic substances among those listed above, which class is distinct from the class(es) to which belong the other(s) anti-ischemic substance combined therewith.

In certain embodiments, the substances having an anti-ischemic effect that are comprised in an anticonvulsive pharmaceutical composition according to the invention consist of antioxidant substances or compounds selected from the group consisting of glutathion, N-acetylcysteine, alpha-lipoic acid, resveratrol (CAS registry n^{o} 501-36-0), ramelteon ((S)—N-[2-(1,6,7,8-tetrahydro-2H-indeno-[5,4-b]furan-8-yl)ethyl]propionamide; CAS Registry no 196597-26-9), a retinoid compound, an antioxidant vitamin, co-enzyme Q-10, beta carotene, uric acid, L-2-oxothiazolidine-4-carboxylic acid and melatonin.

The antioxidant vitamin may be selected from the group consisting of α-tocopherol, vitamin B, vitamin C, vitamin D, vitamin E, vitamin K, and salts thereof.

In certain embodiments, the substances having an anti-ischemic effect that are comprised in an anticonvulsive pharmaceutical composition according to the invention consist of antioxidant compounds selected from the group consisting of dopamine or a precursor or a metabolite thereof, including L-DOPA.

In certain other embodiments, the substances having an anti-ischemic effect that are comprised in an anticonvulsive pharmaceutical composition according to the invention consist of antioxidant compounds selected from the group consisting of a flavonoid, a polyphenol, a phytooestrogen or an extract from Ginkgo biloba.

In certain further embodiments, the substances having an anti-ischemic effect that are comprised in an anticonvulsive pharmaceutical composition according to the invention consist of antioxidant compounds selected from the group consisting of a SOD-like substance and a catalase-like substance.

In yet further embodiments, the substances having an anti-ischemic effect that are comprised in an anticonvulsive pharmaceutical composition according to the invention consist of free radical scavenger substances selected from the group consisting of tirilazad, ebselen, ederavone and melatonin.

In still further embodiments, the substances having an anti-ischemic effect that are comprised in an anticonvulsive pharmaceutical composition according to the invention consist of ACE inhibitors selected from the group consisting of captopril, enalapril, ramipril and lisinopril.

In other embodiments, the substances having an anti-ischemic effect that are comprised in an anticonvulsive pharmaceutical composition according to the invention consist of AT-1 antagonists selected from the group consisting of Losartan, Candesartan, Irbesartan, Valsartan and Telmisartan.

In certain embodiments, the substances having an anti-ischemic effect that are comprised in an anticonvulsive pharmaceutical composition according to the invention consist of calcium channel inhibitors selected from the group consisting of a calcium antagonist, a calcium release blocker agent and a calcium channel blocker.

In further embodiments, the substances having an anti-ischemic effect that are comprised in an anticonvulsive pharmaceutical composition according to the invention consist of calcium channel inhibitors selected from the group consisting of nimodipine, nicardipine, flumarizine, diltiazem, dantrolene, verapamil, nifedipine, and nilvadipine.

In other embodiments, the substances having an anti-ischemic effect that are comprised in an anticonvulsive pharmaceutical composition according to the invention consist of sodium channel blocker agents selected from the group consisting of mexiletine and lidocaine.

In further embodiments, the substances having an anti-ischemic effect that are comprised in an anticonvulsive pharmaceutical composition according to the invention consist of potassium channel activator agents selected from the group consisting of a poly-unsaturated fatty acid, a lysophospholipid, diaxozide, aprikalim, BMS-191095 and NS1619.

In still further embodiments, the substances having an anti-ischemic effect that are comprised in an anticonvulsive pharmaceutical composition according to the invention consist of beta-adrenergic blocking agents selected from the group consisting of solatol, timolol, esmolol, carteolol, carvedilol, nadolol, propanolol, betaxolol, penbutolol, metoprolol, labetalol, acebutolol, atenolol, pindolol, bisoprolol and oxprenolol.

In yet further embodiments, the substances having an anti-ischemic effect that are comprised in an anticonvulsive pharmaceutical composition according to the invention consist of compounds that reduce the extracellular glutamate level and/or prevent the depolarization and overexcitation of postsynaptic cells, e.g. by presynaptically released glutamate. Illustrative examples of such compounds consist of the 2-pyrrolidinone derivatives disclosed in the U.S. Pat. No. 6,984,659

In certain other embodiments, the substances having an anti-ischemic effect that are comprised in an anticonvulsive pharmaceutical composition according to the invention consist of anticonvulsive agents selected from the group consisting of phenyloin and lamotrigine.

In yet further embodiments, the substances having an anti-ischemic effect that are comprised in an anticonvulsive pharmaceutical composition according to the invention consist of NMDA-receptor antagonists such as dextrorphan.

In certain embodiments, the substances having an anti-ischemic effect that are comprised in an anticonvulsive pharmaceutical composition according to the invention consist of hormones selected from the group consisting of estradiol and progesterone.

In other embodiments, the substances having an anti-ischemic effect that are comprised in an anticonvulsive pharmaceutical composition according to the invention consist of vasodilatator agents selected from the group consisting of prostacyclin, cyclic adenosine monophosphate and forskolin.

In further embodiments, the substances having an anti-ischemic effect that are comprised in an anticonvulsive pharmaceutical composition according to the invention consist of α-receptor agonists selected from the group consisting of lisuride, dexmedetomidine, sulpiride, haloperidol, bromocriptine and ropinirole.

In still further embodiments, the substances having an anti-ischemic effect that are comprised in an anticonvulsive pharmaceutical composition according to the invention consist of xanthine oxidase inhibitors or of cyclooxygenase inhibitors selected from the group consisting of allopurinol, oxypurinol and nimesulide.

In yet further embodiments, the at least one substance having an anti-ischemic effect that is comprised in an anticonvulsive pharmaceutical composition according to the invention consist of the protease inhibitor aprotinin.

In other embodiments, the substances having an anti-ischemic effect that are comprised in an anticonvulsive pharmaceutical composition according to the invention consist of immunosuppressant agents selected from the group consisting of cyclosporin A, tacrolimus and a steroid compound

In certain embodiments, the at least one substance having an anti-ischemic effect that is comprised in an anticonvulsive pharmaceutical composition according to the invention consist of a thiazolinedione compound.

In certain other embodiments, the at least one substance having an anti-ischemic effect that is comprised in an anticonvulsive pharmaceutical composition according to the invention consists of a mitochondrial ATP sensitive potassium channel opener agent, for example selected from the group consisting of diazoxide (7-chloro-3-methyl-2H-1,2,4-benzothiazadine-1,1-dioxide), e.g. such as disclosed in the US patent Application no US 2006/0025386

In further embodiments, the at least one substance having an anti-ischemic effect that is comprised in an anticonvulsive pharmaceutical composition according to the invention consist of (2R)—N-(1-benzylpiperidin-4-yl)-3-cyc-lohexylmethylthio-2-((4R)-3-t-butoxycarbonyl thiazolidin-4-ylcarbonylamino)propanamide, e.g. such as disclosed in US Patent Application no US 2004/0067891

In still further embodiments, the substances having an anti-ischemic effect that are comprised in an anticonvulsive pharmaceutical composition according to the invention consist of N,N′-disubstituted guanidines, e.g. such as those disclosed in the U.S. Pat. No. 6,673,557

### Pharmaceutical Compositions and Methods.

Generally, a pharmaceutical composition according to the invention comprises the combination of vigabatrin with at least one anti-ischemic substance and further also one or more physiologically acceptable excipients.

The present invention also concerns methods for preventing or treating convulsive disorders comprising a step of administering (i) a combination of vigabatrin and of at least one anti-ischemic substance or (ii) a pharmaceutical composition as defined above, to an individual in need thereof.

The individuals in need of such treatments encompass those, either adult or child patients, which are susceptible to convulsive disorders, especially epilepsy.

Thus, another object of the present invention consists of a method for preventing or treating convulsive disorders of a patient comprising a step of administering to a patient in need thereof a combination of (i) 4-amino-5-hexenoic acid and (ii) at least one substance having an anti-ischemic effect.

In certain embodiments, the said method comprises a step of administering to a patient in need thereof a pharmaceutical composition that is described in the present specification.

By “physiologically acceptable excipient or carrier” is meant solid or liquid filler, diluent or substance which may be safely used in systemic or topical administration. Depending on the particular route of administration, a variety of pharmaceutically acceptable carriers well known in the art include solid or liquid fillers, diluents, hydrotropes, surface active agents, and encapsulating substances.

Pharmaceutically acceptable carriers for systemic administration that may be incorporated in the composition of the invention include sugar, starches, cellulose, vegetable oils, buffers, polyols and alginic acid. Specific pharmaceutically acceptable carriers are described in the following documents, all incorporated herein by reference: U.S. Pat. No. 4,401,663, Buckwalter et al. issued Aug. 30, 1983; European Patent Application No. 089710, LaHann et al. published Sep. 28, 1983; and European Patent Application No. 0068592, Buckwalter et al. published Jan. 5, 1983. Preferred carriers for parenteral administration include propylene glycol, pyrrolidone, ethyl oleate, aqueous ethanol, and combinations thereof.

Representative carriers include acacia, agar, alginates, hydroxyalkylcellulose, hydroxypropyl methylcellulose, carboxymethylcellulose, carboxymethylcellulose sodium, carrageenan, powdered cellulose, guar gum, cholesterol, gelatin, gum agar, gum arabic, gum karaya, gum ghatti, locust bean gum, octoxynol 9, oleyl alcohol, pectin, poly(acrylic acid) and its homologs, polyethylene glycol, polyvinyl alcohol, polyacrylamide, sodium lauryl sulfate, poly(ethylene oxide), polyvinylpyrrolidone, glycol monostearate, propylene glycol monostearate, xanthan gum, tragacanth, sorbitan esters, stearyl alcohol, starch and its modifications. Suitable ranges vary from about 0.5% to about 1%.

For formulating a pharmaceutical composition according to the invention, the one skilled in the art will advantageously refer to the last edition of the European pharmacopoeia or of the United States pharmacopoeia.

Preferably, the one skilled in the art will refer to the fifth edition “2005” of the European Pharmacopoeia, or also to the edition USP 28-NF23 of the United States Pharmacopoeia.

The weight amount of the combination of active ingredients that is contained in each dose of the pharmaceutical composition of the invention will depend on the molecular weight of said therapeutically active compound as well as on the weight amount that is effective in inhibiting or blocking the convulsive disorder. Effective amounts of vigabatrin that are needed for preventing or treating convulsive disorders are well known from the one skilled in the art.

For determining the appropriate amount of the anti-ischemic substance(s) to be combined with vigabatrin, in a dose of a pharmaceutical composition of the invention, the one skilled in the art may advantageously refer to the effective amounts that are already known or determined in the art for the anti-ischemic substance(s) that is (are) comprised therein.

The present invention is further illustrated by the examples below.

### EXAMPLES

### A. Materials and Methods of the Examples

### A. 1. Vigabatrin Treatment

As described previously Duboc, A., Hanoteau, N., Simonutti, M., Rudolf, G., Nehlig, A., Sahel, J. A., Picaud, S. (2004). “Vigabatrin, the GABA-transaminase inhibitor, damages cone photoreceptors in rats.” Annals of Neurology 55(5): 695-705), Wistar rats Rj Wi IOPS Han were purchased from Janvier Breeding Center (Le Genest St Isle, France) at 7 weeks and acclimatised for 2 weeks. VGB was dissolved in 0.9% NaCl at a concentration of 125 mg/ml. It was injected daily and intraperitonealy at a final dose of 250 mg/kg. To investigate the progression of visual defects with the treatment duration, rats of the same age were divided in five groups and treated with the drug for different times as described in table 1 hereunder.

TABLE 1

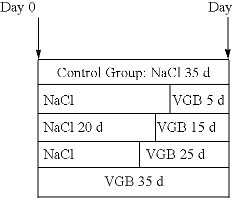

### A.2. Flicker ERG Recording

Flicker ERGs were recorded on each animal before the treatment, at the end of the VGB treatment, and 4-day later. For the study of functional recovery after VGB withdrawal, flicker ERGs were recorded before the treatment, at 35-day of treatment, 16-day recovery and 60-day recovery period.

Animals were kept in the dark room for at least 12 hours and handled under dim red light. They were anaesthetized by an intraperitoneal (i.p) injection (1 to 1.5 ml/kg) of a solution containing ketamine (40 mg/ml) and xylazine (4 mg/ml Rompum). Pupils were dilated with a drop of 0.5% tropicamide, and the cornea was locally anaesthetized with a drop of 0.4% chlorhydrate d'oxybupropacaïne. Measurements were obtained from the right eye of animals laid on a temperature controlled blanket. The measuring electrode, a gold ring covered with a plastic contact lens, was placed on the cornea and a tight electrical contact was obtained by addition of methylcellulose. The reference and ground electrodes made from needles were subcutaneously inserted at the head and the tail respectively. Finally, the whole animal was placed in a Ganzfeld bowl delivering light stimuli (Toennies Multiliner Vision, Höchberg, Germany). For flickering ERG measurements, animals were adapted for 10 minutes to a white background light at a 25 cdm⁻² to suppress rod responses. Flicker responses were obtained with 50 white light flashes at a 2.5 cdsm⁻² intensity and a 15 Hz flickering frequency (High pass filter 1 Hz and low pass filter 300 Hz). Amplitude and implicit time responses of the flicker ERG were measured at the first maximum positive peak with respect to the light onset.

### A.3. Exploration of Fundus Eye and Indocyanine Green (ICG) Angiography

Examination of the eye fundus was performed with a Scanning Laser Opthalmoscope (SLO) (HRA, Heidelberg, Dossenheim, Allemagne). Pupils were dilated with a drop of 0.5% tropicamide. During the examination an experimenter hold the animal to point the eye in the axis of camera. To limit animal stress, the duration of examination was minimized and these short sequences were alternated with rests periods. Eye fundus in each animal eye was examined in unanaesthetized rats with the 534 nm laser light beam using a filter to decrease its intensity. Fundus autofluorescence was obtained with the 488 nm laser beam.

For ICG angiography, animals were anesthetized as above. A solution of Infracyanine (0.1 ml, 5 mg/ml, Société d'Etude et de Recherche Biologiques, Paris, France) dissolved in distilled water was injected intravenously. Images were obtained with the 795 nm laser light immediately after the injection.

### A.4. Tissue Preparation for Immunohistotochemistry

After the animal sacrifice, the cornea was branded at the upper side to orient the eye cup. The eyes were dissected, the cornea and lens removed and a nick was made on the upper side of the eye cup. Eye cups were embedded in OCT (Labonord, Villeneuve d'Ascq, France) to prepare cryosection (Duboc et al 2004, Supra). Dorso-ventral retinal sections passing through the optic nerve were stained with Diamidi-phenyl-indole (DAPI). The DAPI-nuclear staining was visualized on a Leica microscope (20× objective) and digitalized on a camera coupled to optical image analysis software (Analysis program). The retinal length showing displaced photoreceptor nuclei was measured on both part of the optic nerve on the retina of VGB-treated rat.

For immunolabeling, the oriented retinal sections were permeabilized for 5 minutes in PBS containing 0.1% Triton X-100 (Sigma, St Louis, Mo.), rinsed and incubated in PBS containing 1% bovine serum albumin (Eurobio, Les-Ulis, France), 0.1% Tween 20 (Sigma) and 0.1% sodium azide (Merck, Fontenay-Sous-Bois) for 2 hours at room temperature. The primary antibodies added to the solution were incubated for 2 hours at room temperature. Antibodies used were the anti-VEGF rabbit polyclonal antibody (1:400, Ab-1, NeoMarkers, Interchim, France), the monoclonal anti-vimentin antibody (1:400, cloneV9, DAKO, France), the polyclonal mCAR antibody (1:10,000; a gift from Dr Cheryl Craft (Keck School of Medicine of the University of Southern California, Los Angeles, USA)), and the monoclonal anti-G0-α antibody (1:500, Chemicon). After several washes, sections were incubated with the secondary antibodies, goat anti-rabbit IgG conjugated to Alexa TM 594 and goat anti-mouse IgG conjugated to Alexa TM 488 (1:500, (Molecular Probes, Eugene, Oreg.) for 2 hours. The nuclear dye, DAPI, was added in an incubation solution. Sections were mounted with the Fluorsave reagent (Calbiochem, San Diego, Calif.).

### A.5. Statistical Analyses

The statistical analyses on the time dependence of VGB effect were performed by three-way analysis of variance and by a one way analysis of variance for the recovery study, and after applying a Tukey test. The comparison of migration length between the upper and lower retina was tested by a correlation of Pearson test. The cone density was analyzed by a bilateral Student-t-test for two independent populations. For all statistical tests significance was accepted as p<0.05. The statistical analyses were realized under MINITAB software.

### B. Results

### Example 1

### Retinal Functionality

To evaluate the progression of the VGB-elicited damage, flicker ERG were recorded in 4 groups treated for various time and one control group.

Before the treatment, the difference among the groups were not statistically significant (control group NaCl 35 d: 119 μV±17, S.E.M, n=6, VGB 5 d: 90 μV±11, n=5, S.E.M, VGB 15 d: 94 μV±10, n=5, S.E.M, VGB 25 d: 99 μV±9 μV, n=6, S.E.M, and VGB 35 d: 101 μV±7, n=5, S.E.M).

After the injections, flicker ERGs responses were not changed compared for the control group and for the group receiving a 5-day VGB treatment (NaCl 35 d: 113 μV±18 and VGB 5 d: 83 μV±8). By contrast, flicker ERG amplitudes had significantly decreased by 45% in the 15 day VGB-treated rats (52 μV±17, S.E.M), 79% in 25 day VGB-treated rats (21 μV±4) and 71% in the 35 d VGB-treated rats (29 μV±5).

The reduction was statistically significant for the VGB 25 d and VGB 35 d compared with their own flicker responses obtained at d 0 (p<0.001). When compared to the responses measured in the control group at the end of the experiment, the difference were statistically significant for the 15 day treated rats (p<0.01) and the 25 day and 35 day treated animals (p<0.001). These results indicated that the decrease in the amplitude of the flicker response was correlated to the length of the VGB treatment.

Subsequently, the treatment was arrested to allow the drug clearance and the flicker responses were recorded after a 4-day recovery period. For the 15 day VGB-treated group, no change in their flicker response was obtained with respect to the value measured at the end of treatment (51 μV±15, S.E.M) whereas an increase in amplitudes was observed for the 25 day VGB-treated rats (38 μV±4) and the 35 day VGB-treated animals (50 μV±10) but these difference were not statistically significant with the values recorded at the end of the treatment. The difference with the flicker response before the treatment remained statistically significant for the 25 day VGB-treated rats (p<0.01) but lost its significance for the 35 day treatment (p=0.0586). These results indicated that a partial functional recovery is obtained after VGB clearance following long term treatment but that it does not recover to the initial values.

To determine whether a correlation may be established between VGB doses and the visual defect, the cumulative doses were plotted as a function of the amplitude of the flicker responses. The decrease in amplitude of the flicker responses gradually decreased to reach a plateau for the higher doses (2500 mg and 3200 mg). A great correlation was observed between the doses and the functional impairment assessed by the flicker responses (r²=0.90). However, when considering the same animals following the recovery period, a plateau of higher amplitude was already reached at lower doses. These observations suggest that irreversible retinal damage may occur with low doses while higher doses may generate additional but transient functional alterations.

To determine whether larger functional recovery could be obtained with longer recovery periods, flicker ERG were recorded in rats treated with VGB for 35 days immediately, 16 and 60 days after the treatment. As previously the flicker ERGs were reduced by 58% (51 μV±3, S.E.M, n=6) compared with the values at the beginning of experiment (122 μV±5) (p<0.001). At the 16 day and 60 day recovery, the amplitudes of flicker ERGs were less reduced than immediately after the treatment corresponding to 31% (84 μV±5) and 28% (88 μV±10) of the original value measured before the treatment (p<0.001). The results confirm a partial recovery when arresting the treatment. These experiments suggest an irreversible loss of functional retina.

### Example 2

### Retinal and Choroidal Morphology

To determine whether the functional impairment of the retina was associated to visible morphological changes of the retinal tissue, the eye fundus of VGB-treated animals was explored in vivo with the scanning laser opthalmoscope (SLO). The results illustrate the different modifications of the retina that were detected in vivo. First, when the eye fundus was examined using the green laser, some retinal areas appeared clearly modified showing the appearance of a dark/grey network. These modified areas were very extended in the dorsal part of the eye whereas they were limited to discrete spots in the ventral part. Such modified areas were present in all animals examined after a treatment of 25 days (n=4), 35 days (n=1) and 55 days (n=4). They remained visible even after arresting the VGB treatment for one month, in all animal observed (n=2). No such modified areas were detected in control animals of a similar age (n=7) or in animals treated for 5 days (n=1) or 11 days (n=3). When the retinal autofluorescence was examined in vivo with the scanner laser opthalmoscope, fluorescent spots were observed in the modified areas in two rats. The results indicated that some VGB-elicited retinal damage can be observed in vivo with the SLO.

Using SLO, the choroidal vessels were examined in VGB-treated animals by angiography with indocyanine green (ICG). ICG angiograms were collected before the VGB treatment and after a treatment of 25 (n=4) and 55 days (n=4). The results show that no change in the size or aspect of the choroidal vasculature could be detected during the VGB treatment although great modifications could be observed at the eye fundus. After a few minutes, the extravasation of ICG into the choroidal stroma and RPE-Bruch's membrane complex generated a high fluorescent background in the choroids of control animals. By contrast, an inhomogeneous fluorescent image was observed in the areas showing modified eye fundus in animals treated for 55 days. These results indicated that no change in the choroidal vasculature of treated animals could be detected but that the ICG angiography can also be used to investigate the progression of the retinal defects.

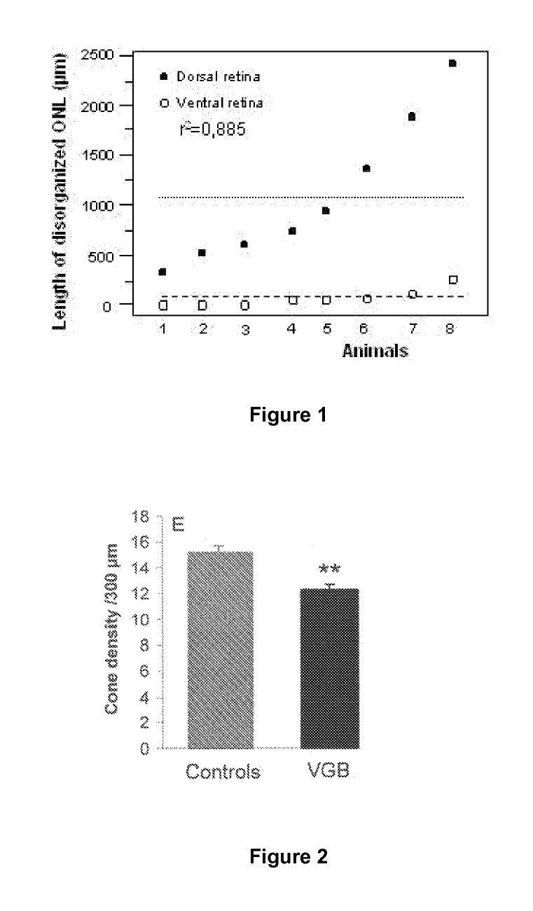

In the SLO examination of the eye fundus, the lesions appeared localized to the upper area of the retina. The inventors therefore investigated whether this distribution of the lesions could be confirmed and quantified on histological sections. Retinal areas with a photoreceptor abutting the retinal pigment epithelium were measured on both sides of the optic nerve in VGB-treated animals following a treatment of 45 days. In all animals, the upper pole exhibited a greater length of disorganized outer retina (mean: 1106 μm±259, S.E.M, n=8) than the lower pole (mean 70 μm±32, S.E.M, n=8). This difference was statistically significant (p<0.001). When animals were ordered by increasing length of disorganized retina in the lower pole, the size of the lesion in the upper pole were similarly increasing gradually (See FIG. 1). A correlation analysis showed that the growth of the lesion in the upper and lower poles were dependant. These results demonstrated that the VGB treatment affected preferentially the upper pole of the rat retina but that although less sensitive, the lower pole was affected proportionally.

To determine whether the cellular damages were restricted to these specific cellular compartment, vertical section of animals treated for 45 days were labelled with a cone arrestin antibody. In control animals, the antibody labelled the entire cell from the outer segment to its terminals. In VGB-treated animals, many cones were observed to be truncated, showing cell bodies deprived of their inner/outer segments. When cellular bodies of cone photoreceptors were quantified following this cone arrestin immunolabeling, the density of cones was observed to decrease by 18.7% in the VGB-treated animals (12.34±0.36 cells/300 μm, E.T, n=7) compared with control rats (15.19±0.51 cells/300 μm, E.T, n=8) (FIG. 2). This difference was statistically significant (P<0.01). When ON bipolar cells postsynaptic to photoreceptors were labeled with G0α antibody, some cells were observed to send a process into the ONL. These observations confirmed that cone photoreceptors were damaged in central areas inducing reorganization in postsynaptic cells.

Since the vigabatrin-induced cone damage and the consecutive reorganization of the postsynaptic cells could result from a metabolic shortage due to a retinal pigment epithelium dysfunction indicated by abnormal ICG angiography, we examined whether it induced the expression of VEGF, a factor induced in hypoxic conditions (Eichler W, Kuhrt H, Hoffmann S, Wiedemann P, Reichenbach A (2000), Neuroport, Vol. 11:3533-3537). In control animals, VEGF was located at the ILM where it colocalized with vimentin-positive glial cells. In the VGB-treated animals, VEGF was also expressed in vertically-oriented processes of Müller glial cells that were immunopositive for vimentin. These VEGF-positive Müller cell processes were not only present in the highly disorganized retinal areas but also in normally appearing areas in the upper and lower poles of the retina. Although Müller cells processes could by immunolabeled for VEGF on their whole length they appeared less frequently labeled in the outer retina. In the VGB-treated rats, vimentin-positive processes of glial cells were occasionally observed to cross the outer limiting membrane (OLM) in normally appearing areas. These observations indicated an increase in VEGF expression following the VGB treatment.

It has been shown in the examples herein that, in VGB-treated rats, the measurement of flicker ERG just after arresting the treatment and few days later indicated a partial recovery of cone visual activity associated with the drug clearance but it confirmed the occurrence of irreversible loss of cone function. The loss of cone photoreceptors was further demonstrated by the immunolabeling of cone photoreceptors with a cone arrestin specific antibody and their subsequent quantification on histological sections. As a consequence of photoreceptor degeneration, postsynaptic ON bipolar cells were seen to send processes in the outer nuclear layer. A major disorganization of the outer nuclear layer which was mainly localized in the upper part of the retina, could be detected in vivo with the scanning laser opthalmoscope. In these areas, the observation of autofluorescent spots was consistent with the occurrence of photoreceptor degeneration. Although no modifications of the choroid vasculature could be detected in vivo as a consequence of the treatment, retinal ischemia was demonstrated by the increase in VEGF expression in glial cells throughout the retina.

1. An anticonvulsive pharmaceutical composition comprising, as the active ingredients, a combination of (i) 4-amino-5-hexenoic acid and (ii) at least one substance having an anti-ischemic effect.

2. The pharmaceutical composition according to claim 1, wherein the at least one substance having an anti-ischemic effect is selected from the group consisting of an antioxidant substance, a free radical scavenger substance, a statin, an ACE inhibitor, an AT-1 antagonist, a calcium channel inhibitor, a sodium channel blocker agent, a potassium channel activator agent, a beta-adrenergic blocking agent, an inhibitor of the synaptic release of glutamate, an antagonist of a glutamate receptor, an anesthetics substance, an anticonvulsive agent, an NMDA-receptor antagonist, a hormone, a vasodilatator agent, an α-receptor antagonist, a xanthine oxidase inhibitor, a cyclooxygenase inhibitor, a protease inhibitor, an immunosuppressant agent and a mitochondrial ATP sensitive potassium channel opener.

3. The pharmaceutical composition according to claim 2, wherein the at least one substance having an anti-ischemic effect consists of an antioxidant compound selected from the group consisting of glutathion, N-acetylcysteine, alpha-lipoic acid, Resveratrol, ramelteon, agomelatin, a retinoid compound, an antioxidant vitamin, α-tocopherol C, D, K and B-complex, co-enzyme Q-10, beta carotene, uric acid, L-2-oxothiazolidine-4-carboxylic acid and melatonin.

4. The pharmaceutical composition according to claim 2, wherein the at least one substance having an anti-ischemic effect consists of an antioxidant compound selected from the group consisting of dopamine or a precursor or a metabolite thereof, including L-DOPA.

5. The pharmaceutical composition according to claim 2, wherein the at least one substance having an anti-ischemic effect consists of an antioxidant compound selected from the group consisting of a flavonoid, a polyphenol, a phytooestrogen or an extract from Ginkgo biloba.

6. The pharmaceutical composition according to claim 2, wherein the at least one substance having an anti-ischemic effect consists of an antioxidant compound selected from the group consisting of a SOD-like substance and a catalase-like substance.

7. The pharmaceutical composition according to claim 2, wherein the at least one substance having an anti-ischemic effect consists of a free radical scavenger substance selected from the group consisting of tirilazad, ebselen, ederavone and melatonin.

8. The pharmaceutical composition according to claim 2, wherein the at least one substance having an anti-ischemic effect consists of an ACE inhibitor selected from the group consisting of captopril, enalapril, ramipril and lisinopril.

9. The pharmaceutical composition according to claim 2, wherein the at least one substance having an anti-ischemic effect consists of an AT-1 antagonist selected from the group consisting of Losartan, Candesartan, Irbesartan, Valsartan and Telmisartan.

10. The pharmaceutical composition according to claim 2, wherein the at least one substance having an anti-ischemic effect consists of a calcium channel inhibitor selected from the group consisting of a calcium antagonist, a calcium release blocker agent and a calcium channel blocker.

11. The pharmaceutical composition according to claim 10, wherein the at least one substance having an anti-ischemic effect consists of a calcium channel modifier selected from the group consisting of nimodipine, nicardipine, flumarizine, diltiazem, dantrolene, verapamil, nifedipine, and nilvadipine.

12. The pharmaceutical composition according to claim 2, wherein the at least one substance having an anti-ischemic effect consists of a sodium channel blocker agent selected from the group consisting of mexiletine and lidocaine.

13. The pharmaceutical composition according to claim 2, wherein the at least one substance having an anti-ischemic effect consists of a potassium channel activator agent selected from the group consisting of a poly-unsaturated fatty acid, a lysophospholipid, diaxozide, aprikalim, BMS-191095 and NS1619.

14. The pharmaceutical composition according to claim 2, wherein the at least one substance having an anti-ischemic effect consists of a beta-adrenergic blocking agent selected from the group consisting of solatol, timolol, esmolol, carteolol, carvedilol, nadolol, propanolol, betaxolol, penbutolol, metoprolol, labetalol, acebutolol, atenolol, pindolol, bisoprolol and oxprenolol.

15. The pharmaceutical composition according to claim 2, wherein the at least one substance having an anti-ischemic effect consists of an anticonvulsive agent selected from the group consisting of phenyloin and lamotrigine.

16. The pharmaceutical composition according to claim 2, wherein the at least one substance having an anti-ischemic effect consists of an NMDA-receptor antagonist consisting of dextrorphan.

17. The pharmaceutical composition according to claim 2, wherein the at least one substance having an anti-ischemic effect consists of a hormone selected from the group consisting of estradiol and progesterone.

18. The pharmaceutical composition according to claim 2, wherein the at least one substance having an anti-ischemic effect consists of a vasodilatator agent selected from the group consisting of prostacyclin, cyclic adenosine monophosphate and forskolin.

19. The pharmaceutical composition according to claim 2, wherein the at least one substance having an anti-ischemic effect consists of an α-receptor agonist selected from the group consisting of lisuride, dexmedetomidine, sulpiride, haloperidol, bromocriptine and ropinirole.

20. The pharmaceutical composition according to claim 2, wherein the at least one substance having an anti-ischemic effect consists of a xanthine oxidase inhibitor or of a cyclooxygenase inhibitor selected from the group consisting of allopurinol, oxypurinol and nimesulide.

21. The pharmaceutical composition according to claim 2, wherein the at least one substance having an anti-ischemic effect consists of the protease inhibitor aprotinin.

22. The pharmaceutical composition according to claim 2, wherein the at least one substance having an anti-ischemic effect consists of an immunosuppressant agent selected from the group consisting of cyclosporine A, tacrolimus and a steroid compound.

23. The pharmaceutical composition according to claim 1, wherein the at least one substance having an anti-ischemic effect consists of a mitochondrial ATP sensitive potassium channel opener selected from the group consisting of diazoxide and cromakalim.

24. The pharmaceutical composition according to claim 1, wherein the at least one substance having an anti-ischemic effect is selected from the group consisting of a thiazolidinedione and a N,N′-disubstituted guanidine.

25. A method for preventing or treating convulsive disorders of a patient comprising a step of administering to a patient in need thereof a composition a combination of (i) 4-amino-5-hexenoic acid and (ii) at least one substance having an anti-ischemic effect.

26. The method according to claim 25, wherein the at least one substance having an anti-ischemic effect is selected from the group consisting of an antioxidant substance, a free radical scavenger substance, a statin, an ACE inhibitor, an AT-1 antagonist, a calcium channel inhibitor, a sodium channel blocker agent, a potassium channel activator agent, a beta-adrenergic blocking agent, an inhibitor of the synaptic release of glutamate, an antagonist of a glutamate receptor, an anesthetics substance, an anticonvulsive agent, an NMDA-receptor antagonist, a hormone, a vasodilatator agent, an α-receptor antagonist, a xanthine oxidase inhibitor, a cyclooxygenase inhibitor, a protease inhibitor, an immunosuppressant agent and a mitochondrial ATP sensitive potassium channel opener.

27. The method according to claim 25, wherein the convulsive disorder consists of epilepsy.
